(19) United States
(12) Patent Application Publication
(10) Pub. No.: US 2004/0241088 A1
(43) Pub. Date: Dec. 2, 2004

(54) Method for evaluating the efficacy of certain cancer treatments

(75) Inventors: Esther H. Chang, Potomac, MD (US); Kathleen F. Pirollo, Rockville, MD (US); Antonina S. Rait, Rockville, MD (US)

Correspondence Address: ROTHWELL, FIGG, ERNST & MANBECK, P.C., 1425 K STREET, N.W., SUITE 800, WASHINGTON, DC, 20005, US

(73) Assignee: Georgetown University, Washington, DC

(21) Appl. No.: 10/765,568

(22) Filed: Jan. 28, 2004

Related U.S. Application Data
(60) Provisional application No. 60/442,902, filed on Jan. 28, 2003.

(51) Int. Cl. A61K 51/00

(52) U.S. Cl. 424/1.11

(57) ABSTRACT

A method for evaluating the efficacy in the body of a mammal of a therapeutic agent which acts to stimulate apoptosis comprises:

obtaining a sample of a body tissue in which tumor cells are present or a body fluid from a mammal to be treated with the therapeutic agent which acts to stimulate apoptosis, wherein the tissue or fluid can contain a 17 kDa fragment of Caspase 3, the fragment obtained by specific cleavage of Caspase 3 in vivo;

assaying the sample to determine the amount of the 17 kDa fragment of cleaved Caspase 3 present;

administering the therapeutic agent to the mammal;

obtaining a second sample of said body tissue or body fluid from said mammal; and

assaying the second sample to determine the amount of said 17 kDa fragment of cleaved Caspase 3 present;

wherein an increase in the amount of the 17 kDa fragment measured in the second sample over the amount measured in the first sample indicates apoptosis stimulation and efficacy of the therapeutic agent.

This application claims priority from U.S. Provisional Application 60/442,902, filed Jan. 28, 2003.

### FIELD OF THE INVENTION

This invention relates to a method for evaluating the efficacy of cancer therapies that act through or result in the induction of apoptosis. More specifically, this invention relates to such a method which involves detecting the 17 kDa subunit of cleaved Caspase 3 as a marker of apoptosis.

2. Description of the Background Art

One highly productive focus of research in recent years in the development of systemic gene therapy delivery systems has been on developing non-viral, pharmaceutical formulations of genes for in vivo human therapy, particularly cationic liposome-mediated gene transfer systems (Massing U, et al., Int. J. Clin. Pharmacol. Ther. 35:87-90 (1997)). Features of cationic liposomes that make them versatile and attractive for DNA delivery include: simplicity of preparation; the ability to complex large amounts of DNA; versatility in use with any type and size of DNA or RNA; the ability to transfect many different types of cells, including non-dividing cells; and lack of immunogenicity or biohazardous activity (Felgner P L, et al., Ann. NY Acad. Sci. (1995) 772:126-139; Lewis J G, et al., Proc. Natl. Acad. Sci. USA (1996) 93:3176-3181). More importantly from the perspective of human cancer therapy, cationic liposomes have been proven to be safe and efficient for in vivo gene delivery (Aoki, K. et al., Cancer Res. 55:3810-3816 (1997); Thierry, A. R., Proc. Natl. Acad. Sci. USA 92:9742-9746 (1997)).

The transfection efficiency of cationic liposomes can be dramatically increased when they bear a ligand recognized by a cell surface receptor. Receptor-mediated endocytosis represents a highly efficient internalization pathway present in eukaryotic cells (Cristiano, R. J., et al., Cancer Gene Ther. 3:49-57 (1996), Cheng, P. W., Hum. Gene Ther. 7:275-282 (1996)). The presence of a ligand on a liposome facilitates the entry of DNA into cells through initial binding of the ligand by its receptor on the cell surface followed by internalization of the bound complex. A variety of ligands have been examined for their liposome-targeting ability, including transferrin and folate (Lee, R. J., et al., J. Biol. Chem. 271:8481-8487 (1996)). Folate receptor (FR) and Transferrin receptor (TfR) levels are elevated in various types of cancer cells including, but not limited to, prostate, breast, pancreatic, head and neck, bladder, brain, ovarian, skin, lung, and liver cancers. Elevated TfR and FR levels also correlate with the aggressive or proliferative ability of tumor cells (Elliot, R. L., et al., Ann. NY Acad. Sci. 698:159-166 (1993)). Therefore, TfR and FR levels are considered to be useful as prognostic tumor markers, and they are potential targets for drug delivery in the therapy of malignant cells (Miyamoto, T., et al., Int. J. Oral Maxillofac. Surg. 23:430-433 (1994); Thorstensen, K., et al., Scand. J. Clin. Lab. Invest. Suppl. 215:113-120 (1993)).

In addition to ligands that are recognized by receptors on tumor cells, specific antibodies also can be attached to the liposome surface (Allen, T. M., et al., Stealth Liposomes, pp. 233-244 (1995)), enabling them to be directed to specific tumor surface antigens (including but not limited to receptors) (Allen, T. M., Biochim. Biophys. Acta 1237:99-108 (1995)). These “immunoliposomes,” especially the sterically stabilized immunoliposomes, can deliver therapeutic drugs to a specific target cell population (Allen, T. M., et al., Stealth Liposomes, pp. 233-244 (1995)). Park, et al. (Park, J. W., et al., Proc. Natl. Acad. Sci. USA 92:1327-1331 (1995)) found that anti-HER-2 monoclonal antibody (Mab) Fab fragments conjugated to liposomes could bind specifically to HER-2 overexpressing breast cancer cell line SK-BR-3. The immunoliposomes were found to be internalized efficiently by receptor-mediated endocytosis via the coated pit pathway and also possibly by membrane fusion. Moreover, the anchoring of anti-HER-2 Fab fragments enhanced their inhibitory effects. Doxorubicin-loaded anti-HER-2 immunoliposomes also showed significant and specific cytotoxicity against target cells in vitro and in vivo (Park, J. W., et al., Proc. Natl. Acad. Sci. USA 92:1327-1331 (1995)). In addition, Suzuki et al., Br. J. Cancer 76:83-89 (1997), used an anti-transferrin receptor monoclonal antibody conjugated immunoliposome to deliver doxorubicin more effectively in human leukemia cells in vitro. Huwyler et al., Proc. Natl. Acad. Sci. USA 93:14164-14169 (1996), used an anti-TfR monoclonal antibody immunoliposome to deliver daunomycin to rat glioma (RT2) cells in vivo. This PEGylated immunoliposome resulted in a lower concentration of the drug in normal tissues and organs. These studies demonstrated the utility of immunoliposomes for tumor-targeting drug delivery.

Progress in biotechnology has allowed the derivation of specific recognition domains from Mab (Poon, R. Y., Biotechnology International: International Developments in the Biotechnology Industry, pp. 113-128 (1997)). The recombination of the variable regions of heavy and light chains and their integration into a single polypeptide provides the possibility of employing single-chain antibody derivatives (designated scFv) for targeting purposes. Retroviral vectors engineered to display scFv directed against carcinoembryonic antigen, HER-2, CD34, melanoma associated antigen and transferrin receptor have been developed (Jiang, A., et al., J. Virol. 72:10148-10156, (1998); Konishi, H., et al., Hum. Gene Ther. 9:235-248 (1994); Martin, F., et al., Hum. Gene Ther. 9:737-746 (1998)). These scFv directed viruses have been shown to target, bind to and infect specifically the cell types expressing the particular antigen. Moreover, at least in the case of the carcinoembryonic antigen, scFv was shown to have the same cellular specificity as the parental antibody (Nicholson, I. C., Mol. Immunol. 34:1157-1165 (1997)).

A variety of immunoliposomes are capable of tumor-targeted, systemic delivery of nucleic acids for use in human gene therapy. The antibody- or antibody fragment-targeted immunoliposome complexes can be made via chemical conjugation of the antibody or antibody fragment to the liposome complex or by a simple and efficient non-chemical conjugation method. The TfRscFv can be chemically conjugated to lipoplex using various methods (PCT application publication No. WO 00/50008, incorporated herein by reference) and can efficiently transfect human prostate tumor cells in vitro and in vivo. Alternatively, the antibody or single chain protein is bound to the liposome and the antibody- or scFv-liposome-therapeutic or diagnostic agent complex is formed by simple mixing of the antibody or scFv, liposome and ligand in a defined ratio and order.

The targeted liposomes can carry a variety of therapeutic molecules to target cells. Therapeutic agents are attached to the liposome surface. Such agents include chemotherapeutic agents, high molecular weight DNA molecules (genes), plasmid DNA molecules, and small oligonucleotides.

To date, more than 600 gene therapy clinical trials have been approved worldwide, and this number will only increase. Currently, however, there is a dearth of reliable, minimally invasive means of determining if a particular gene therapy is reaching, and affecting, its intended target. This issue is of particular importance as research moves toward the development of systemic gene therapy delivery systems that can affect metastatic disease. Since repeated tumor biopsies are not practicable, it is imperative to develop new approaches that employ less invasive methodologies.

Many cancer agents, including, but not limited to, chemotherapeutic agents, radiation, and tumor suppressor genes, such as RB and p53, work by inducing apoptosis. Apoptosis (also called programmed cell death) is a highly regulated physiological process that plays a central role in tissue patterning during development and in maintaining homeostasis in adult cells/tissue (Horvitz, H. R., Cancer Res. 59:1701-1706 (1999); Jacobsen, M. D. and Weil, M., Cell, 88:407-454 (1997)). Defects in the apoptotic machinery are a hallmark of cancer (Hanahan, D., and Weinberg, R., Cell, 100:57-70 (2000)).

It is an object of this invention to provide a simple and reliable and less-invasive method for evaluating the efficacy of a therapeutic agent in the body of a mammal. More particularly, it is an object of the present invention to provide such a method when the therapeutic agent acts to stimulate apoptosis.

### SUMMARY OF THE INVENTION

In accordance with this invention, a method is provided for evaluating the efficacy in the body of a mammal of a therapeutic agent which acts to stimulate apoptosis. The method comprises:

obtaining a sample of a body tissue in which tumor cells are found or a body fluid from a mammal to be treated with said therapeutic agent, wherein said tissue or fluid can contain a 17 kDa fragment of caspase 3, said fragment obtained by specific cleavage of caspase 3 in vivo;

assaying said sample to determine the amount of said 17 kDa fragment of caspase 3 present;

administering said therapeutic agent to said mammal;

obtaining a second sample of said body tissue or body fluid from said mammal; and

assaying said second sample to determine the amount of said 17 kDa fragment of caspase 3 present;

wherein an increase in the amount of said 17 kDa fragment measured in said second sample over the amount measured in said first sample correlates with apoptosis stimulation by and efficacy of said therapeutic agent.

### BRIEF DESCRIPTION OF THE FIGURES

FIG. 1 shows the 17 kDa cleaved caspase 3 subunit from mouse plasma purified through P30 and P6 columns.

FIG. 2 shows the expression of exogenous wtp53 and 17 kDa caspase 3 subunit expression in Panc-1 xenografts following i.v. injection of TfRscFv-LipA-p53.

FIG. 3 shows the expression of the 17 kDa caspase 3 subunit in Panc-1 xenografts over time following i.v. injection of a complex of folate-LipA-p53. In the Figure, UT=untreated animal used as a control; Fpp53=folate-Liposome A-p53 complex; and FpVec=folate-Liposome A complex carrying an empty vector.

FIG. 4 shows the presence of the 17 kDa protein in blood cell pellets extracted from DU145 tumor-bearing mice following treatment with a complex of transferrin-liposome A-p53 and cisplatin (CDDP).

FIG. 5 shows the presence of the 17 kDa subunit of caspase 3 in serum from mice with or without PANC-1 xenograft tumors following treatment with a combination of a complex comprising transferrin-liposome A-p53 and cisplatin.

FIG. 6 shows the presence of the 17 kDa subunit of caspase 3 in PANC-1 cells following treatment with a complex of TfRscFv-liposome A-antisense HER-2 in comparison to such cells treated with a complex of TfRscFv-liposome A-scrambled HER-2.

FIG. 7 shows the presence of the 17 kDa subunit of caspase 3 in PANC-1 cells following treatment with a combination of the TfRscFv-liposome A-antisense HER-2 complex and Gemzar® in comparison to untreated cells and to treatment with either Gemzar® alone, the TfRscFv-liposome A-AS HER-2 complex alone, or the combination of TfRscFv-liposome A-scrambled HER-2 complex and Gemzar®.

FIG. 8 shows the presence of the 17 kDa subunit of caspase 3 in plasma from mice bearing PANC-1 xenograft tumor following i.v. administration of a combination of a complex of TfRscFv-liposome A-antisense HER-2 and Gemzar® in comparison to an untreated animal or to treatment with either Gemzar® alone, the TfRscFv-liposome A-AS HER-2 complex alone or the combination of a complex of TfRscFv-liposome A-scrambled HER-2 and Gemzar®.

FIGS. 9A and 9B show in vitro down-modulation of protein expression in apoptotic pathways by TfRscFv-liposome A-antisense HER-2 alone or in combination with Gemzar® eight hours post-transfection of PANC-1 and COLO357 cells, respectively. Both types of cells showed clear evidence of the presence of the 17 kDa subunit of caspase 3. These results are contrasted to the results in untreated cells and in cells which were treated with either Gemzar® alone or a combination of Gemzar® and TfRsvFv-liposome A-scrambled HER-2.

FIG. 10 shows in vitro down-modulation of protein expression in apoptotic pathways by TfRscFv-liposome A-antisense HER-2 alone or in combination with Gemzar® sixteen hours post-transfection of PANC-1 cells. Controls as in FIGS. 9A and 9B.

FIG. 11 shows the localization of the antisense HER-2 effect in tumor cells following i.v. delivery of TfRscFv-LipA-antisense HER-2 complex alone or in combination with Gemzar® into nude mice bearing subcutaneous PANC-1 xenograft tumors. The arrow showing the presence of the 17 kDa subunit points to the middle band in the tumor that is not present in either the liver or lung cell samples.

FIG. 12 is a graft showing the in vivo effect of the combination of TfRscFv-liposome A-antisense HER-2 and Gemzar® treatment on PANC-1 xenograft tumors in comparison to untreated tumors or tumors treated with Gemzar® alone, the complex alone or a combination of Gemzar® and a complex of TfRscFv-liposome A-scrambled HER-2.

FIG. 13 shows the presence of the 17 kDa subunit of caspase 3 in mouse plasma following systemic treatment with the RB94 tumor suppressor gene.

FIG. 14 shows the presence of the 17 kDa subunit of caspase 3 in serum of human breast cancer patients after chemotherapy.

### DETAILED DESCRIPTION OF THE INVENTION

The mechanism of apoptosis is remarkably conserved throughout evolution and is controlled by a family of cysteine proteases. These enzymes cleave after an asparate residue in their specific substrate, thus mediating many of the typical biochemical and morphological changes that characterize apoptotic cells. While it is known to one well versed in the field that identification of activated caspases can be used as an indicator or biochemical marker of apoptosis, their use up to now has been only in in vitro cell culture or lysate or in intact live cells.

There are at least 14 different mammalian members of the caspase family. These enzymes are constitutively expressed in most cell types as inactive precursors (zymogens) that undergo proteolytic activation in response to proapoptotic signals (see Kohler et al., J. Immuno Methods 265:97-110 (2002)). The large proenzyme is cleaved at specific internal sequences separating the large and small subunits which then form a heterodimer (Jacobsen, M. D. and Weill, M; Cell 88:307-354 (1997); Cryns, V. and Yuan, M. J., Gene Dev. 12:1551-1570 (1998); and Nunez, G., et al., Oncogene 17:3237-3245 (1998)). The active caspase is composed of two such heterodimers (Nicholson, D. W. and Thornberry, N., trands biochem. Sci. 22:299-306 (1997)).

The caspases involved in apoptosis generally are divided into two categories, the initiator caspases (caspases 2, 8, 9 and 10) and the effector caspases (caspases 3, 6 and 7). The former group autoactivate, then proceed to activate the effector caspases. It is these activated effector caspases that cleave a spectrum of cellular targets ultimately leading to cell death. This sequential activation of initiator to effector caspases has lead to the idea of a caspase cascade. For example, binding of tumor necrosis factor (TNF) or fas ligand to its receptor leads to the assembly of the “death-inducing signaling complex” which recruits initiator pro-caspase 8 resulting in its activation. Active caspase 8 cleaves and activates pro-caspase 3 giving rise to the proteolytic cascade.

In healthy cells, the caspases exist in mitochondria and cytosol as their inactive proenzymes (Mancini, M., et al., J. Cell Biol. 140: 1485-1495 (1998)). Apoptotic signals are transduced along two major pathways: an intrinsic pathway associated with the mitochondria and an extrinsic pathway mediated by death receptors of the tumor necrosis factor receptor superfamily. This cascade can be triggered by a number of different types of stimuli (Mathiasen and J\ddot{aa}ttelä, Trends in Molecular Medicine 8:212-20 (2002)). Agents that damage DNA, such as irradiation and chemotherapeutic agents, activate p53, which can stimulate both pathways of apoptosis. Importantly, caspase 3 activation is required for the execution of both pathways. Thus, caspase 3-induced proteolysis has been shown to be a critical event in virtually all cellular apoptotic pathways. All of the current data suggests that defects in apoptosis are a prerequisite of cancer (J\ddot{aa}ttelä, M., Exp. Cell Research 248:30-43 (1999); Evans, G. and Vousden, K., Nature 411:342-348 (2001). Cell growth signals induced by unregulated activity of oncoproteins, such as HER-2, or inactivation of tumor suppressor proteins, such as p53, should trigger caspase activation and increase apoptosis. However, human tumors contain mutations in pro-apoptotic genes (leading to their inactivation) (e.g. p53) and/or have increased expression/activity of anti-apoptotic proteins (Mathiason and J\ddot{aa}ttelä TRENDS in Mol. Med. 8:212-220 (2002)) (e.g. HER-2), resulting in a reduction of or inability of a tumor cell's ability to respond to therapeutic modalities.

It now has been found that detection of the 17 kDa subunit of caspase 3 in tumor cells and/or a body fluid provides a means of verifying the efficacy of therapy by showing that the apoptotic pathway is functioning following administration of a therapeutic agent that acts to stimulate apoptosis. Thus, by measuring the level of the 17 kDa subunit in a sample of a body fluid or tumor cell-containing body tissue from a patient prior to initiation of treatment with such a therapeutic agent, and comparing that level to the level of the 17 kDa subunit in a second sample of the body fluid or tumor cell-containing body tissue from the patient following treatment with the therapeutic agent, one can determine whether the therapeutic agent has stimulated apoptosis.

The method of this invention can be used to both qualitatively measure the existence of apoptosis and to evaluate the extent of apoptosis. Thus, the method can be used in a dose response study to compare and evaluate the relative effectiveness of different therapies. The more effective a particular therapy is, the higher the level of apoptosis and, therefore, the greater the amount of the 17 kDa subunit that will be produced. In accordance with this invention, apoptosis as a result of the action by a therapeutic agent or combination of therapeutic agents will be found to have occurred if the amount of the 17 kDa subunit of caspase 3 in the tumor cells or body fluid is found to be at least about 1.5-2 times above any background level (i.e., of the amount of the subunit measured in a sample of the tumor cells or body fluid from the same host prior to the administration of the therapeutic agent(s)). A highly efficacious therapeutic regimen can result in 17 kDa levels at least about 3 to 4 times that of any background level. If the tissue or body fluid sample obtained prior to administration of the therapeutic agent shows no presence of the 17 kDa subunit, then any amount of the subunit detected in the second sample, obtained post-administration of the therapeutic agent, is viewed as a result of the action of the agent inducing apoptosis.

Typically, measurement of the amount of the 17 kDa subunit in a tumor sample or body fluid sample can be carried out from about 30 minutes to about 5 days following administration of the therapeutic agent, depending upon the nature of the agent, and preferably from about 8 hours to about 72 hours post-administration. The administration of a therapeutic agent comprising a HER-2 antisense oligonucleotide, for example, results in apoptosis relatively rapidly, whereas a therapeutic agent comprising a wtp53 gene takes longer to be effective. If the treatment is a multi-dose treatment spread over a number of days or weeks, one can determine the amount of the subunit 30 minutes—5 days following the initial treatment, after each treatment or following the last of the treatments. If the treatment is effective, the amount of apoptosis and, therefore, the amount of the 17 Da subunit produced will keep increasing over time.

As noted above, amounts of the 17 kDa subunit can be measured in either tumor cells or a body fluid. The body fluid can comprise blood or a component thereof, such as serum or plasma, or saliva. The preferred body fluid is blood or a component thereof.

This method of evaluating the efficacy of a particular therapy is effective with any therapeutic agent or modality which acts to stimulate apoptosis. Such agents include irradiating or radiotherapeutic agents, chemotherapeutic agents and tumor suppressor genes such as p53, RB 94 and RB or oligonuceotides, such as antisense HER-2 or a combination thereof, such as the administration of a tumor suppressor gene in combination with radiation or chemotherapy. Preferred agents include a DNA molecule encoding a wild type p53 molecule, an RB or RB94 molecule, an apoptin molecule and a HER-2 antisense oligonucleotide.

In one embodiment, the therapeutic agent comprises a gene therapy and is administered via a viral vector, or, more preferably, as part of a cationic liposome-ligand complex, as described above. Such complexes are described in detail in U.S. patent application Ser. Nos. 09/601,444; 09/914,046 and 10/113,927 incorporated herein by reference in their entireties. These complexes are targeted to a site of interest, typically to a cancer cell, such as a cancer cell expressing a transferrin receptor. The targeting agent is the ligand, such as transferrin or folate or an antibody or antibody fragment, which binds to a receptor of interest on the target cells. A preferred antibody fragment is a single chain Fv fragment (scFv). Such a fragment contains the complete antibody binding site for the epitope of interest recognized by the intact antibody and is formed by connecting the component VH and VL variable domains from the heavy and light chains, respectively, with an appropriately designed linker peptide which bridges the C-terminus of one variable region and N-terminus of the other, ordered as either VH-linker-VL or VL-linker-VH. The therapeutic complexes can be administered intratumoraly, intraperitonealy, intramuscularly, orally or systemically, preferably intravenously.

It now has been found that there is an association between exogenous expression of certain therapeutic agents, such as wtp53, RB94 or antisense HER-2 and changes in angiogenic and apoptotic factors in vivo. The tumor's response correlates to the administration of the therapeutic agent, and these changes show that the factors can serve as useful molecular markers for the effectiveness of the therapy in treating cancer. When tumor and tissue samples are obtained, the extent of apoptosis can be determined using various known method of analysis, such as a Western blot assay, the TUNNEL assay or the AnnexinV Staining (TREVIGEN TACS®) apoptosis detection kit. The presence of the 17 kDa cleaved caspase 3 subunit can be assessed by Western analysis in tissues and in blood samples. Correlation of changes with the presence of the therapeutic agent, such as exogenous wtp53 expression, in the tumor and tumor response supports the use of the caspase 3 subunit as a marker of tumor response.

In specific preferred therapeutic treatments, the therapeutic composition comprises either a nucleic acid encoding p53, RB or RB94 or an antisense (AS) HER-2 oligonucleotide. It is known that the apoptotic pathway is induced by p53 RB or RB94, and it now has been found that it also is induced by AS HER-2 treatment. It has been shown that through its interaction with the P13K/Akt pathway, HER-2 can affect apoptosis, and down-modulation of HER-2 following administration of an antisense HER-2 oligo induces caspase 3 cleavage. One example of a ligand-liposome-antisense HER-2 complex is a TfRscFv-lipA-AS HER-2 complex, wherein TfRscFv stands for a single chain Fv fragment of a monoclonal antibody which binds to the transferrin receptor and LipA represents a cationic liposome comprising a 1:1 ratio of dioleoyltrimethylammonium phosphate (DOTAP) and dioleoylphosphatidylethanolamine (DOPE). This and similar complexes are described in detail in U.S. patent application Ser. No. 09/914,046, incorporated herein by reference. Examples of ligand-cationic liposome-p53 complexes are described in detail in U.S. patent application Ser. No. 09/601,444, incorporated herein by reference, and include DOTAP:DOPE, DOTAP:cholesterol, DOTAP:DOPE:cholesterol, dimethyldioctadecylammonium bromide (DDAB):DOPE, DDAB:cholesterol or DDAB:DOPE:cholesterol. As illustrated in the Examples below, there is a clear correlation between treatment with wtp53, RB94 or AS HER-2 and the presence of the 17 kDa subunit as a marker of apoptosis.

The level of the 17 kDa subunit of caspase 3 can be measured by obtaining either samples of the tumor or of the patient's blood both before and after treatment with the selected therapeutic composition. It has been found that this subunit is not detectable in blood cell pellets or in tumor cells from untreated tumor-bearing subjects or from normal cells from tumor-bearing subjects but is detectable in both the blood and in tumor cells following treatment with therapeutic agents which induce apoptosis. As noted above, a measurement of the 17 kDa subunit which is at least about 1.5-2 times the background amount is indicative of apoptosis resulting from the action of the therapeutic agent administered.

Expression of the 17 kDa subunit of caspase 3 can be determined using a commercially available antibody to the fragment, such as one from Cell Signaling Technology, Beverly, Mass., by Western analysis.

By measuring the levels of the 17 kDa fragment, one can evaluate and establish the efficacy of a therapy of interest. For example, as described in detail in the examples below, the effects of treatment with a combination of TfRscFv-liposome-AS HER-2 and the chemotherapeutic agent Gemzar® (gemcitabine) on induction of the 17 kDa fragment in mice bearing human pancreatic cancer xenograft tumors who had received multiple i.v. treatments of the antibody fragment-liposome complex carrying either antisense HER-2 or a scrambled HER-2 oligonucleotide plus multiple treatments of Gemzar® were determined. Animals receiving either Gemzar® alone or the antibody fragment-liposome-antisense HER-2 oligonucleotide complex alone were used as controls. Western analysis of serum samples clearly indicated a synergistic induction of the 17 kDa subunit in animals treated with the antisense-containing liposome complex plus Gemzar® in comparison to treatment with either therapy alone. This strong induction was not evident in mice receiving the scrambled oligo-containing liposome complex plus Gemzar®. This and the other studies described in detail in the Examples demonstrate that the 17 kDa subunit can be used as a non-invasive pharmacodynamic marker for therapeutic efficacy.

### EXAMPLES

### Example 1

### Method of Isolating Protein from Cell Culture and Tissue

A. To detect the presence of the 17 kDa active caspase 3 fragment in cell cultures the following procedure was used in subsequent Examples to isolate total protein from both living and dead floating cells. The medium from the cell culture vessel was removed and reserved.

Procedure:

Wash the cells in the vessel (e.g. 75 cm² flask) with 10 ml cold PBS. Combine the PBS with reserved medium. Add 5 ml PBS to each flask and scrape the cells with a rubber policeman. Add the cells to the media/PBS solution. Add 5 ml PBS to each flask and wash. Check under the microscope to determine amount of cells remaining. If necessary, add 5 ml PBS and scrape again. Add to the previous solution.

Centrifuge at 200-300×g for 7 minutes at 4° C.

Remove supernatant from the tube, leaving cell pellet intact.

Resuspend the cell pellet in 1 ml of PBS and transfer to a 1.5 ml microcentrifuge tube.

Centrifuge again, as above.

Remove PBS, leaving cell pellet intact.

To lyse the cells, resuspend the cell pellet in 150 μl-200 μl RIPA buffer (with freshly prepared inhibitors) per 3×10⁶ cells. RIPA buffer is 1×PBS, 1% NP40, 0.5% sodium deoxycholate, 0.1% SDS (this may be made in large volumes). Add inhibitors at time of use from the following stock solutions:

a) 10 mg/ml PMSF in isopropanol (use at 10 μl/ml).

b) Aprotinin (Sigma catalog # A6279, use at 30 μl/ml).

c) 100 mM sodium orthovanadate in frozen aliquots (use at 10 μl/ml).

Incubate the cell suspension on ice for 20 minutes, vortex every 5-7 minutes (avoid generating bubbles in the solution).

Pass 5-10 times through a 21½ gauge needle. Add freshly prepared PMSF and incubate on ice for an additional 20 minutes.

Centrifuge at 13,000×g for 10 minutes at 4° C.

Transfer supernatant (cell lysate) to a 1.5 ml microcentrifuge tube before the pellet dissociates.

Aliquot 30-50 μl cell lysate per tube and freeze at −70° to −80° C. Make one 5-10 μl aliquot for use in determining protein concentration using the Pierce Micro BCA protein assay kit, with BSA as the standard, according to the manufacturer's protocol.

B. The following procedure was used in the experiments of subsequent Examples to isolate total protein from tumor or any other animal organ/tissue. After euthanasia the tissues were rapidly dissected from the animal, rinsed in excess cold PBS 1-3 times and minced while kept on ice using clean, sterile instruments. The minced tissue was placed in one or more sterile pre-weighed polypropylene tubes and immediately flash frozen in liquid nitrogen by immersion. The frozen tissue was kept at −70° C. to −80° C. until ready to isolate protein.

Procedure:

Place tissue on dry ice, and place a clean, sterile Bessman tissue pulverizer into liquid nitrogen for a few minutes before use.

Place tissue in cold pulverizer and crush by hammering to a very fine powder.

Quickly harvest the pulverized tissue (powder) using a clean, dry, cold spatula, and place in a microcentrifuge tube on dry ice. Rapidly weigh tube and tissue to get approximate weight of tissue.

To lyse tissue, add 600 μl RIPA buffer (with freshly added inhibitors) for every 100 μg tumor tissue, or 1 ml of RIPA buffer for every 100 μg of normal tissue. Vortex to mix and follow above procedure for cell culture.

### Example 2

### Preparation of Plasma for Analysis of Caspase 3 17 kDa Fragment in Blood

In the procedures set forth in subsequent Examples, either of two methods were used to collect blood for preparation of plasma to be used for assessment of the caspase 3 17 kDa fragment.

In one preferred embodiment whole blood was taken from an animal or a human in standard heparinized 3 ml tubes (Vacutainer®, CAT#366387, Becton Dickson VACUTAINER® Systems, Franklin Lakes, N.J.) containing 45 USP units of Sodium Heparin, mixed well and placed on ice. For small blood volumes 30 μl of 1× PBS was added to the 3 ml VACUTAINER® tube to dissolve the Heparin and 1/25 to 1/50 ratio of Heparin/Blood volume desired was placed in a sterile microcentrifuge tube. To this tube 50-100 μl of fresh blood was added, mixed well and placed on ice. The blood/Heparin mixture was centrifuged at 1000×g at 4° C. for 10 minutes (large volumes were transferred from the VACUTAINER® tube to a sterile microcentrifuge tube prior to centrifugation). After centrifugation the plasma was removed and placed into a separate sterile microcentrifuge tube. The plasma could be aliquoted and frozen at −70°-−80° C.

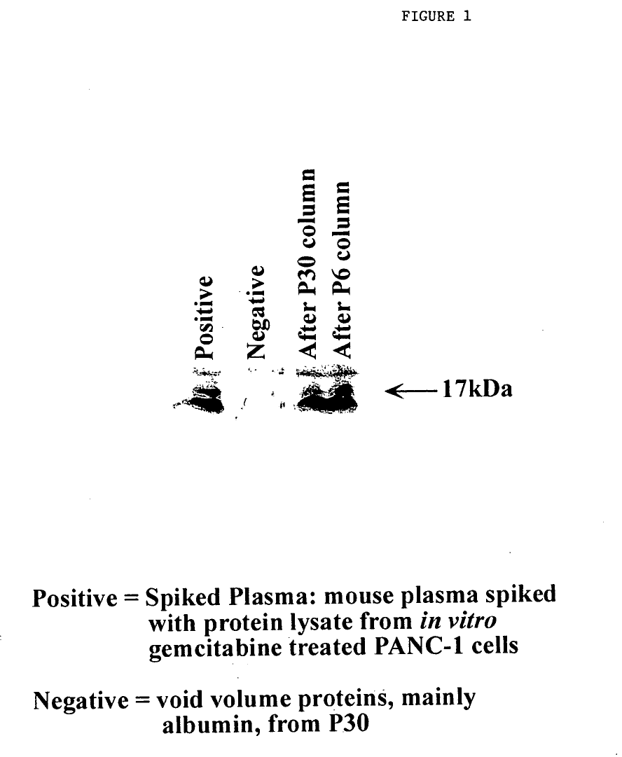

In another preferred embodiment, whole blood was collected in heparinized tubes and plasma obtained as above. To remove other blood components that might interfere with detection of the 17 kDa fraction the plasma could be purified using the commercially available “Micro Bio-Spin”® Chromotography Columns (Bio-Rad Laboratories, Hercules Calif.). Either the P6 column (in Tris) or the P30 column (in Tris) could be used. However, in the preferred embodiment P6 (in Tris) was used. The plasma was purified according to the manufacturer's protocol except that in one embodiment before Step 2 (centrifuging the column to remove the remaining packing buffer) the column was washed once by gravity with 1 ml of 10 mM Tris-HCl buffer pH=7.4-8.0 without sodium azide. The 17 kDa protein was in the flow through. FIG. 1 shows the 17 kDa cleaved caspase 3 fragment purified in this manner from P30 and P6 columns. The positive control was unpurified mouse plasma spiked with protein lysate from PANC-1 cells treated in vitro with gemcitabine which induces apoptosis. The negative controls were void volume proteins, mainly albumin, from a P30 column using gravity flow rather than centrifugation.

As an alternative to plasma, serum could also be isolated from blood. In this case no Heparin was used. Instead, the whole blood was allowed to coagulate in a non-Heparinized tube at room temperature for 30 minutes to 1 hour then the samples were centrifuged at 0.1×g for 10 minutes and the serum removed. Serum could also be stored at −70° to −80° C. and also can be purified by the P6 or P30 Microspin columns as described above.

### Example 3

### Western Analysis of Proteins for Caspase 3 17 kDa Expression

Western analysis of proteins from blood or tissue was performed as follows:

Electrophoresis

Dilute the total protein cell lysate (20-60 μg total protein) with an equal volume of RIPA buffer at 4° C. Mix well.

Mix this lysate solution with 1/6 volume of 6× electrophoresis sample buffer [6× Electrophoresis sample buffer (for discontinuous systems): 7ml of 4× Tris-HCl, pH 6.8, 3.0 ml glycerol, 1 g SDS, 0.93 g DTT, 1.2 mg bromophenol blue, add H₂O to 10 ml (if needed). Store in 0.5 ml aliquots at −70° C.

Boil for 5 minutes, then pulse at 13,000 ×g for 5 seconds at room temperature.

Load immediately onto a Criterion precast 4-20% gel from Bio-Rad Laboratories (Hercules, Calif.) or any appropriate gel such as a 13% polyacrylamide gel, a 4-20% polyacrylamide/SDS gel. Run gel at 100 V, but not higher than 30 MA, until the dyefront appears at the bottom of the gel.

Alternatively, after column chromatography on Mini Bio-Spin P-30 or P-6 columns, up to 10 μl of human serum will be separated on NuPAGE® Bis-Tris Electrophoresis Systems using 4-12% or 10-20% precast gels and MES SDS running buffer (Invitrogen, Life Technologies, Carlsbad, Calif.) according to the manufacturer's protocol.

Sample Preparation:

10 μl of human serum (after P-30 or P-6 purification) is diluted to 1× sample buffer with 4× sample buffer (Invitrogen) and the same solution the post-column sample is in to a total volume of 24 μl (for 1 mm combs) and 36 μl for 1.5 mm combs). NuPAGE® Reducing Agent (0.5 M DTT in stabilized liquid form) is added to 10% of the final sample volume just prior to heating the solution at 65-75° C. (preferably 70° C.) for 5-15 minutes, preferably 10 minutes, and loading the sample onto the NuPAGE® gel. MES SDS Running buffer is used and the gel is run at 100-200 constant voltage and 70-125 m A per gel.

Transfer, immunoblotting and detection are performed as described above.

sample buffer

| | |
|---|---|
| NuPAGE ® LDS sample buffer (4×) | 10 ml |
| glycerol | 4.00 g |
| Tris base | 0.682 g |
| Tris HCl | 0.666 g |
| LDS | 0.800 g |
| EDTA | 0.006 g |
| Serva Blue G250 | 0.75 ml of 1% solution |
| Phenol Red | 0.25 ml of 1% solution |
| Ultrapure water to | 10 ml |

1× buffer should be pH 8.5
no acid or base should be used to adjust pH.
LDS = lithium dodecyl sulfate

Reducing Conditions:

dilute 20× NuPAGE® SDS Running Buffer (MES) to prepare 1× NuPAGE® SDS Running Buffer as follows:

Mix thoroughly:

| | |
|---|---|
| NuPAGE ® SDS Running Buffer (MES) (20×) | 50 ml |
| ultrapure water | 950 ml |

Set aside 800 ml of the 1× NuPAGE® SDS Running Buffer for use in the lower (outer) buffer chamber. Approximately 600 ml of 1× Running Buffer will fill the lower buffer chamber. Immediately prior to the run, prepare the remaining 200 ml for the upper (inner) buffer chamber by adding 500 μl of the NuPAGE® antioxidant per 200 ml 1× NuPAGE® SDS Running Buffer. Mix thoroughly.

MES SDS Running Buffer:

| | |
|---|---|
| (20×) 500 ml MES | 97.6 g (1.00 M) |
| 2-(N-morpholino)ethane sulfonic acid | |
| Tris Base | 60.6 g (1.00 M) |
| SDS | 10.0 g (69.3 mM) |
| EDTA | 3.0 g (20.5 mM) |
| ultrapure water to | 500 ml |

1× buffer should be pH 7.3.
No acid or base should be used to adjust the pH.

### Example 4

### Immunoblotting for Detection of Caspase 3 17 kDa Protein from Blood or Tissue

Immunoblotting

Block non-specific protein binding by soaking the membrane in Blotto A [Blotto A (for general use): 5% (w/v) powdered milk, TBS, 0.05% Tween-20.] for 1 hour. If the entire Western cannot be completed in one day, the membrane should be soaked overnight covered in TBS (without Tween-20), at 4° C.

Incubate the membrane in primary antibody. The primary antibody used is a rabbit polyclonal antibody against cleaved caspase 3 (Asp 175) from Cell Signaling Technology (Beverly, Mass.) (cat# 966115) at a dilution of 1:500 to 1:2000, preferably 1:1000 in 5% (w/v) powdered milk in TBST at a volume of 5-50 ml solution/50 cm² membrane, preferably 10-30 ml/50 cm² preferably overnight at 4° C. with gentle rocking. Alternatively, the primary antibody, at the same dilution, can be incubated at room temperature (20-27° C.) for 2-3 hours with gentle rocking.

Wash membrane with TBST, three times for 10 minutes each wash with gentle rocking.

Incubate with Horseradish Peroxidase (HRP) conjugated goat anti-rabbit, anti-mouse, or anti-rat IgG (secondary antibody), diluted 1:1000 to 1:10,000, preferably 1:2000 in Blotto A, for 30 minutes with gentle rocking.

Wash with TBST three times with gentle rocking for 15 minutes each wash, followed by one wash in TBS for 15 minutes.

Detection is performed using Amersham ECL reagents according to manufacturer specifications.

### Example 5

### Detection of Cleaved Caspase 3 17 kDa Proteins in Pancreatic Tumors and Liver

As one therapeutic agent, a TfRscFv-LipA-p53 complex, described in detail in U.S. patent application Ser. No. 09/914,046, incorporated herein by reference, was used. Athymic nude mice carrying human pancreatic cancer (PANC-1) subcutaneous xenograft tumors were i.v. injected with TfRscFv-LipA-p53 three times over a 24 hour period. For each injection the complex carried 40 μg of p53 plasmid DNA in a total volume of 800 μl/mouse. Sixty hours after the last injection the animals were euthanized, the tumor and liver excised and protein isolated for Western analysis as in Examples 1, 3 and 4. The protein from the tumor and liver of an untreated animal also was included as a control. The same membrane was subsequently probed with a commercial antibody specific for Actin to assess equal loading.

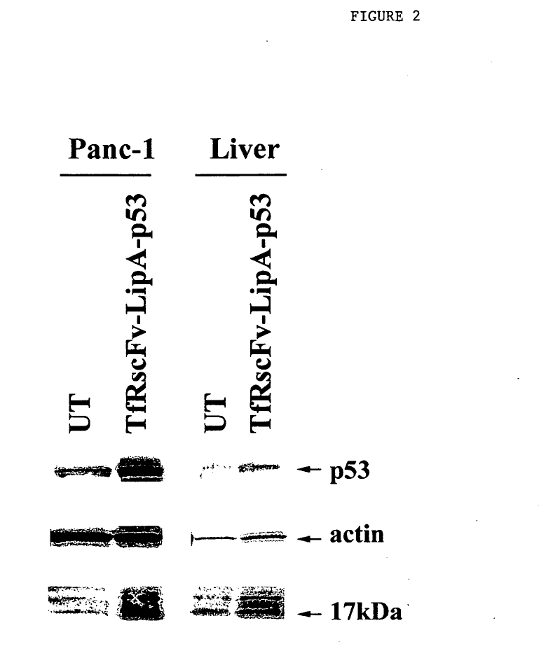

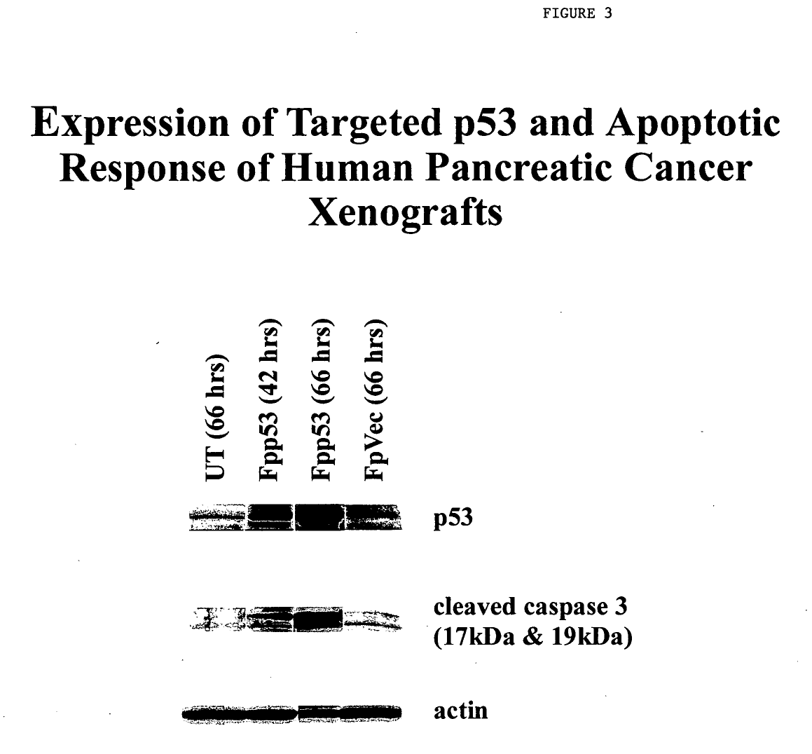

FIG. 2 shows the expression of exogenous wtp53 primarily in the PANC-1 tumors of mice that had been i.v. injected with the TfRscFv-LipA-p53 complex. The identical Western blot also was used to probe for the presence of the 17 kDa fragment. As is shown in the bottom lane of FIG. 3, there was a substantial increase in the presence of this 17 kDa marker protein in the tumor but only low levels evident in the liver after treatment with the TfRscFv-LipA-p53 complex, indicating that restoration of wtp53 function resulted in an induction of apoptosis particularly in the tumor. There was a clear correlation between expression of the exogenous wild-type (wt) p53 and the expression of the 17 kDa cleaved caspase 3 fragment. The upper band of this panel represents a 19 kDa precursor of the 17 kDa subunit.

Ligand-liposomes carrying wtp3, directed by other targeting moieties, showed the same effect. Another study was done using folate as the targeting ligand for the liposome-p53 complex in PANC-1 tumors. Athymic nude mice carrying human PANC-1 subcutaneous xenograft tumors were i.v. injected 3 times within 24 hours with LipA-p53 targeted by a folate ligand. For each injection 20 μg of plasmid DNA/mouse, either carrying the wtp53 DNA, or as empty vector, was included in the complex (total volume of 300 μg/mouse/injection). 42 and 66 hours later the animals were euthanized; the tumor excised and protein isolated for Western analysis as in examples 1, 3 and 4. The protein from the tumor of an untreated animal also was included as a control. The same membrane was subsequently probed with a commercial antibody specific for the 17 kDa subunit of Caspase 3, with an Actin for equal loading. No expression of the 17 kDa fragment was evident in tumors of animals injected with the complex carrying empty vector in place of p53, indicating that this is a p53 directed phenomenon (FIG. 3).

### Example 6

### Detection of 17 kDa Fragment of Cleaved Caspase 3 in Blood Cells

It was envisioned that if the 17 kDa fragment is detectable in blood, it could be used as a non-invasive means of establishing the efficacy of wtp53 gene therapy. To evaluate this, blood was taken from mice systemically treated with Tf-LipA-p53 which has Tf as the targeting ligand. Tf-LipA-p53 (100 μg p53) was i.v. administered once to athymic nude mice bearing Dul45 xenograft tumors over 100 mm³. The mice also received one injection at 5 mg/kg of the chemotherapeutic agent cisplatin (CDDP) (i.p.). Sixty hours later the mice were euthanized and approximately 1 ml. of blood collected in heparinized tubes. The cells were separated by centrifugation, protein isolated from the cell pellets as in Example 1 and run on a 13% polyacrylamide gel as in Example 3. The 17 kDa cleaved active subunit of caspase 3 was identified by Western analysis using an anti-17 kDa specific antibody (Cell Signaling) as described in Example 4.

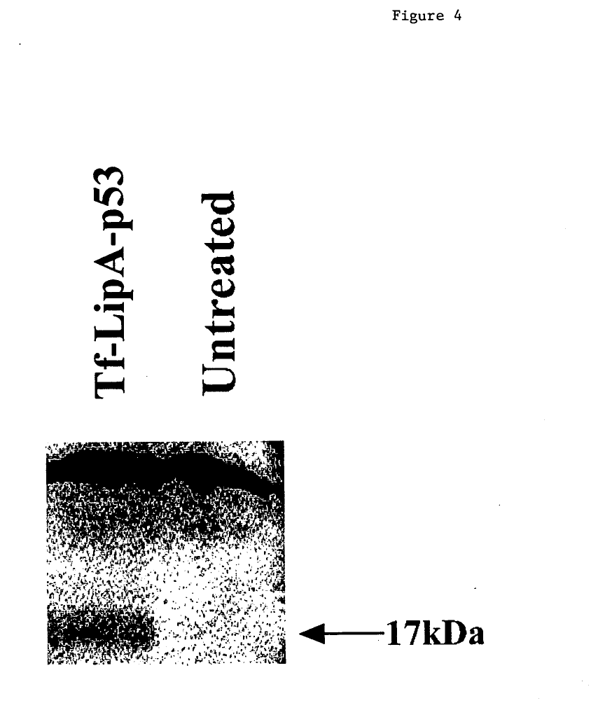

Results in FIG. 4 show the presence of the 17 kDa protein in blood cell pellets extracted from DU145 tumor bearing mice 60 hours after systemic treatment with Tf-LipA-p53. However, this subunit was not detectable in blood cell pellets from the untreated tumor bearing animals. Therefore, this indicates that there is a clear correlation between the presence of exogenous wtp53 and the presence of this marker of apoptosis detectable in blood through relatively non-invasive means.

### Example 7

### Demonstration that Expression of the 17 kDa Fragment of Cleaved Caspase 3 is Related to Tumor Response to Therapy

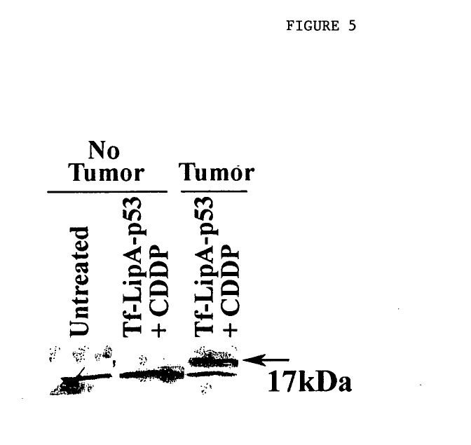

The results in Example 6 show the presence of the 17 kDa protein in blood cell pellets extracted from DU145 tumor bearing mice 60 hours after systemic treatment with Tf-LipA-p53 plus CDDP. Normal lymphocytes are sensitive to p53-induced apoptosis. Therefore, an evaluation was made to determine whether the appearance of the 17 kDa fragment in blood is truly tumor related. The same treatment given to the mice bearing DU145 tumors described in Example 6 was repeated in mice with or without PANC-1 subcutaneous xenograft tumors. Moreover, in this experiment serum was used to try and avoid complications due to the presence of blood cells. Since serum is being used it is not possible to use a housekeeping gene to assess equal loading, but equal volumes were loaded/lane. The serum was isolated from 1 ml of whole blood without use of heparin as described in Example 2 and Western Analysis was performed as described in Examples 3 and 4. As shown in FIG. 5, the 17 kDa fragment was strongly expressed in the tumor-bearing, and only the tumor-bearing, animals. Thus, the presence of this band is clearly related to tumor response to the wtp53/CDDP therapy.

### Example 8

### Detection of the 17 kDa Fragment of Caspase 3 as an Indicator of Apoptosis: After Treatment with AS-HER-2 ODN in Vitro

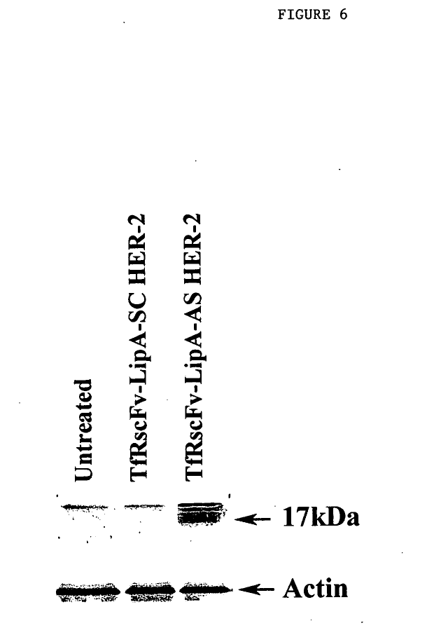

In a further specific embodiment of this invention, it was determined that the cleaved 17 kDa subunit can be detected in tumor and/or blood as a means of verifying the efficacy of therapy with antisense (AS) HER-2, i.e. that the apoptotic pathway is induced by AS HER-2 treatment. (The antisense Her-2 oligonucleotide used is that described in U.S. Pat. No. 6,027,892 and U.S. application Ser. No. 09,716,320, incorporated by reference herein in their entireties. It has been shown that through its interaction with the P13K/Akt pathway HER-2 can affect apoptosis. Thus, it was expected that down-modulation of HER-2 via the TfRscFv-LipA-AS HER-2 complex would induce caspase 3 cleavage. The commercially available (Cell Signaling Technology) antibody to the 17 kDa fragment was used to detect its expression by Western analysis as in Examples 3 and 4. Protein lysates were obtained using the procedure described in Example 1. 24 hours post-transfection, protein from cells treated with the TfRscFv-lipA complex carrying either the AS HER-2 or the scrambled (SC) HER-2 ODN was isolated as described in Example 1. The scrambled HER-2 ODN has the same nucleotide composition as the antisense molecule but in random order. In one embodiment the AS-HER-2 ODN is a 15 nucleotide piece of DNA having homology near the initiation codon to the sense strand of the gene coding for human HER-2 gene. 1.2×10⁶ PANC-1 cells were seeded in a T75 flask and transfected 24 hours later with the TfRscFv-LipA complex containing 0.5 μM of AS HER-2 or scrambled (SC) ODN. 24 hours later, protein was isolated for Western analysis as described in Example 1. 40 μg were loaded/lane of a 4-20% gradient polyacrylamide/SDS gel. After transfer to nitrocellulose membrane, the blot was probed with a commercial Ab specific for the 17 kDa fragment of caspase 3, and Actin for equal loading as in Examples 3 and 4. The band above the 17 kDa band represents a 19 kDa precursor of the 17 kDa subunit. As shown in FIG. 6, there was a clear induction of the caspase 3 17 kDa fragment, demonstrating a stimulation of the apoptotic pathway after TfRscFv-LipA-AS HER-2 treatment. This band was not evident in either the untreated or SC HER-2 ODN treated cells, indicating a clear AS HER-2 specific effect.

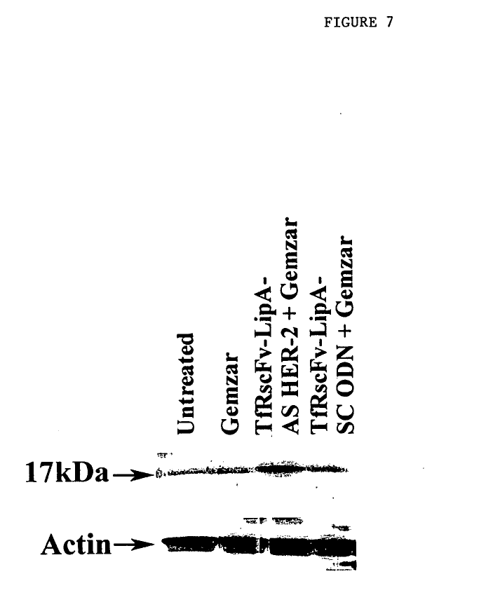

Also evaluated in vitro was the induction of the 17 kDa fragment by the combination of the TfRscFv-LipA-AS HER-2 plus the chemotherapeutic agent Gemzar® (gemcitabine). As shown in FIG. 7, treatment (as above) of PANC-1 cells for 9 hours with 0.8 μM Gemzar® did not result in the expression of this fragment above background levels. In contrast, treatment with TfRscFv-LipA-AS HER-2 (at 1 μM ODN) plus Gemzar® (0.8 μM) induced a strong 17 kDa band which was not present in cells treated with the complex carrying the same amount of SC ODN in combination with the same amount of Gemzar®. This indicates that this was not a non-specific ODN or Gemzar® effect. Actin levels showed equal protein loaded per lane.

### Example 9

### Detection of the 17 kDa Fragment of Cleaved CasPase 3 In Vivo after Treatment with AS HER-2: An In Vivo Pharmacodynamic Marker

The 17 kDa protein also can be used as a non-invasive in vivo pharmacodynamic marker for establishing the efficacy of AS HER-2 therapy. The effects of combination treatment (TfRscFv-LipA-AS HER-2 plus Gemzar®) on induction of the 17 kDa fragment in mice bearing PANC-1 xenograft tumors that had received multiple (a total of 19) i.v. treatments of TfRscFv-LipA, carrying either AS HER-2 or SC ODN (9 mg/kg) plus 11 i.p. injections of Gemzar® (60 mg/kg) were determined. Animals receiving either Gemzar® or the TfRscFv-LipA AS HER-2 complex alone were used as controls.

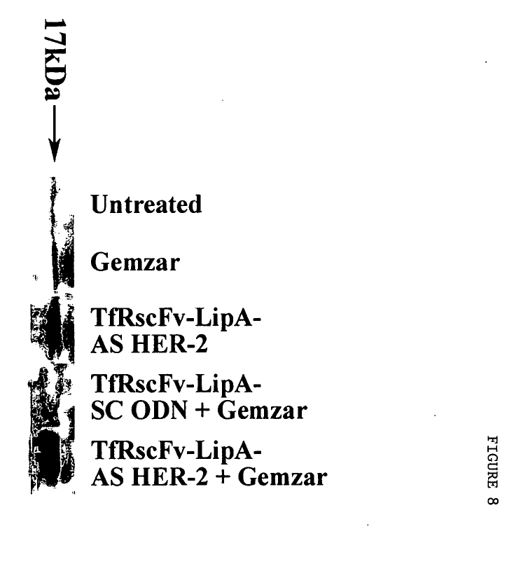

Plasma was isolated from 1 ml of blood from each animal as described above in Example 2. 30 μl of each plasma sample were run on a 4-20% gradient polyacrylamide/SDS gel. The 17 kDa cleaved active subunit of caspase 3 was identified by Western analysis as described in Examples 3 and 4. Western analysis of plasma samples clearly indicated a synergistic induction of the 17 kDa fragment in animals treated with TfRscFv-LipA AS HER-2 plus Gemzar® compared to treatment with either therapy by itself (FIG. 8). This strong induction was not evident in the mice receiving SC ODN (TfRscFv-LipA-SC ODN) plus Gemzar®. There thus is a clear correlation between treatment and effect of either wtp53 or AS HER-2 and the presence of this marker of apoptosis. These studies demonstrate that this protein can be used as a non-invasive pharmacodynamic marker for therapeutic efficacy.

### Example 10

### Expression of the 17 kDa Fragment of Cleaved Caspase 3 in Relation to AS HER-2 in Vitro Down-Modulation of Signal Transduction Pathways

Treatment of Pancreatic Cancer (PanCa) with the tumor targeting TfRscFv-LipA-AS HER-2 complex can down-regulate HER-2 expression (even when not overexpressed), thus negatively affecting cell growth/survival and positively enhancing apoptotic pathways leading to increased tumor cell killing. To demonstrate that HER-2 down-regulation via the TfRscFv-liposome complex can affect down-stream cell signaling pathways the ability of this complex to affect components of the PI3K/AKT pathway and apoptosis in PanCa cell lines PANC-1 and COLO357 was assessed by Western analysis. These two cell lines were chosen because they have different levels of HER-2 expression; COLO357 expresses significantly higher HER-2 levels than PANC-1.

The phosphorothioate sequence specific AS HER-2, complementary to the initiation codon region (5′-TCC ATG GTG CTC ACT-3′), and the control, non-sequence specific SC (5′-CTA GCC ATG CTT GTC-3′) ODNs were synthesized and purified by reverse phase HPLC by Ransom Hill Biosciences (Ramona, Calif.). Screening of both the AS and SC sequences against the GenBank Database indicated that the AS ODN had homology only to HER-2, while there was no homology between the SC ODN and any sequence in the database. PANC-1 or COLO 357 cells were seeded/in a six well plate and transfected 24 hours later with the TfRscFv-LipA complex carrying 1 μM (for PANC-1) or 0.5 μM (for COLO 357) AS HER-2 or SC HER-2 ODN (negative control). The cells were transfected with either oligo alone or, to look for a synergistic effect, in combination with gemcitabine (Gemzar®). At the indicated times, the cells were harvested, lysed in RIPA buffer, protein determined, run (60 ug total protein/lane) on a 4-20% gradient polyacrylamide/SDS gel and transferred to nitrocellulose for Western analysis as described in Examples 1, 3 and 4. To detect HER-protein expression the membranes were probed with the anti-human HER-2/Neu (C-18) rabbit polyclonal Ab (Santa Cruz Biotechnology) and the signal detected by ECL (Amersham). Change in protein expression as compared to untreated cells was also ascertained for total and/or phosphorylated Akt (Ser 473), a central component in the PI3K pathway (using an anti-Human polyclonal Ab, Cell Signaling Technology),phosphorylated BAD (Ser 136), an important factor in regulation of apoptosis (using an anti-human rabbit polyclonal antibody, Cell Signaling Technology), as well as cleaved caspase 3 (Asp 175) (using the rabbit polyclonal antibody, specific for the 17 kDa subunit, Cell Signaling Technology) and PARP/cleaved PARP (poly ADP ribopolymerase, another marker of apoptosis) using an anti-human rabbit polyclonal antibody, (Cell Signaling Technology) both downstream indicators of apoptosis.

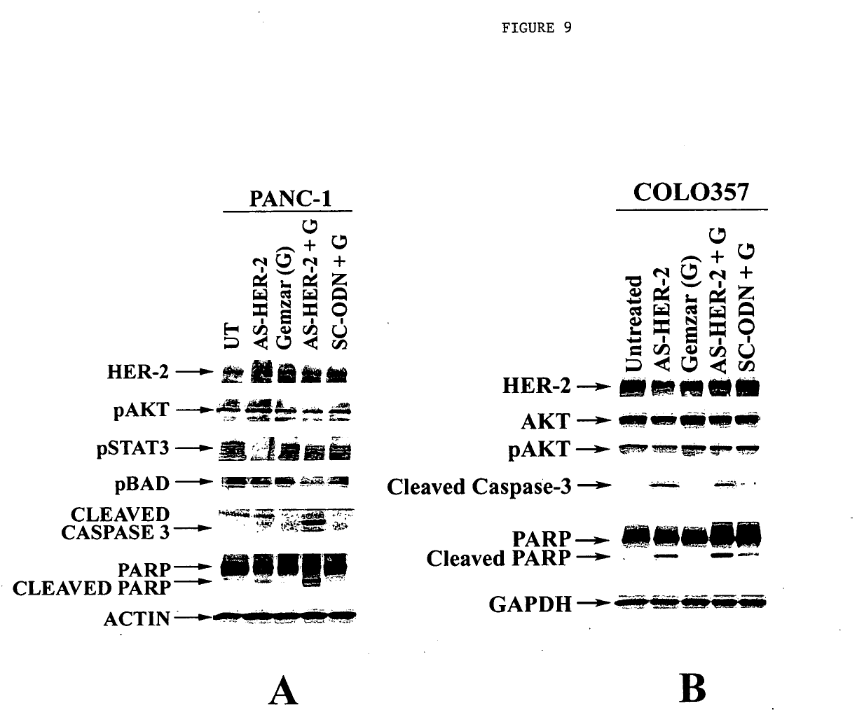

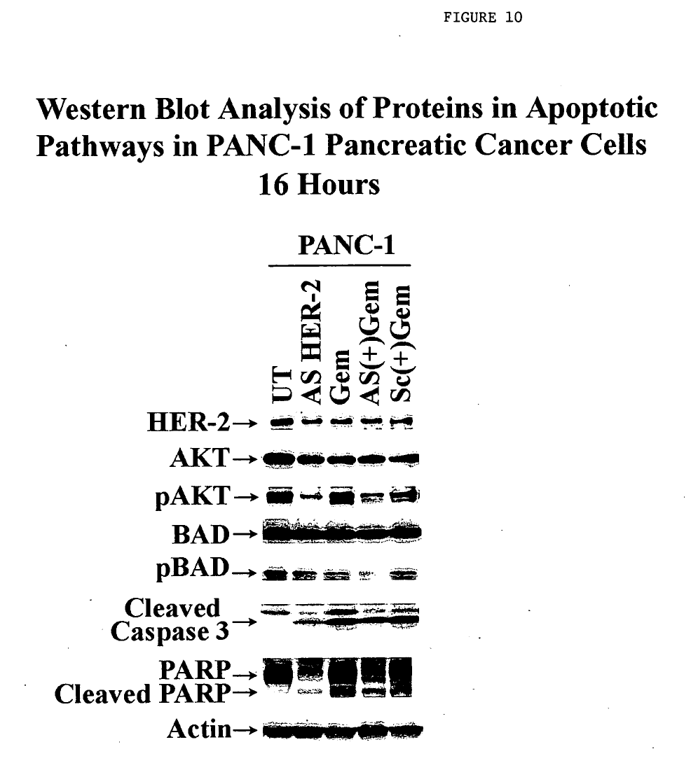

FIGS. 9A and 9B show the effect of transfection of TfRscFv-LipA-AS HER-2, alone or in combination with Gemzar®, eight hours post-transfection. The half-life of the HER-2 protein has been reported to be between 10 and 25 hours (Bae et.al Experimental and Molecular Medicine 33:15-19 (2001)). Thus, as expected, no changes in HER-2 protein levels by Western analysis were detected at this early time. However, this time point was chosen in an effort to detect early antisense specific effects or any synergistic effect of the combination of AS HER-2 plus Gemzar®. In the PANC-1 cells (FIG. 10A), there was some effect on the phosphorylated, active form of AKT by the combination of AS HER-2 and Gemzar®. However, a clear synergistic down-modulation by AS HER-2 plus Gemzar® was evident on the expression of pBAD even at this early time. More significantly, the cleaved forms of caspase 3 (appearance of the 17 kDa protein) and PARP, both indicative of the induction of apoptosis, appeared only in the cells treated with the AS HER-2 ODN, primarily in the combination therapy (but faintly with the single therapy as well) and not in those cells treated with Gemzar® alone or with SC ODN plus Gemzar®, indicating that these effects are AS HER-2 specific. COLO357 cells were also examined for changes in protein expression 8 hrs post-transfection. As observed with PANC-1, at this point in time there was virtually no change in HER-2 expression and only minimal down modulation of pAKT. However, here also both cleaved caspase 3 (17 kDa subunit) and cleaved PARP are clearly evident in both the cells treated with AS HER-2 alone and in combination with Gemzar®. This, taken together with the fact that there is little or no evidence of these bands in the cells treated with Gemzar® only or the combination of SC ODN plus Gemzar®, again demonstrate that this is an AS specific effect.

Since at 8 hours the phosphorylated active form of AKT (pAKT) showed only minimal effect of TfRscFv-LipA-AS HER-2 treatment, PANC-1 cells also were examined 16 hours post-transfection (FIG. 10). As this time point was still less than the reported half-life of the HER-2 protein, there was no HER-2 down-modulation evident, as expected. However, significant down modulation of pAKT was observed in the cells treated with AS HER-2, both alone and in combination with Gemzar®, that was not evident in the controls. pBAD is even further down-modulated at 16 hrs as compared to 8 hrs, its protein expression almost totally eliminated. A Gemzar® effect also now was observed. The cleaved forms of caspase 3 (17 kDa fragment) and PARP were evident not only in the antisense treated cells but also in those treated with Gemzar® alone or with SC ODN plus drug. However, with respect to PARP, there was still a significant difference between the cells treated with AS HER-2 ODN and the controls. In both AS HER-2 single and combination treatment the overall level of PARP (cleaved and uncleaved) was much less than that observed with Gemzar® or SC ODN plus Gemzar®, presumably due to earlier onset and continued degradation as a result of AS HER-2 treatment. It should also be noted that for both AKT and BAD the inactive, unphosphorylated forms of these proteins were unaffected by AS HER-2 treatment, supporting the idea that the observed down-modulation is pathway specific and not a result of general non-specific cytotoxicity of the treatment.

### Example 11

### Detection of the Tumor Specific Localization of the 17 kDa Fragment of Cleaved Caspase 3 in Vivo after Systemic Treatment of PanCa Tumors by AS HER-2

It has been shown that through its interaction with the PI3K/Akt pathway HER-2 can inhibit apoptosis. Thus, down-modulation of HER-2 via the TfRscFv-LipA-AS HER-2 complex should induce caspase 3 cleavage.

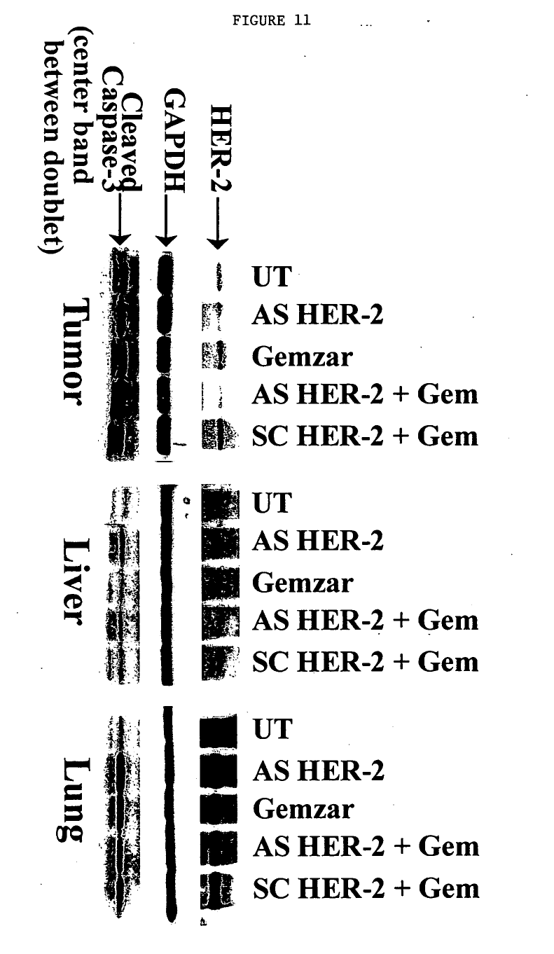

PANC-1 tumors were induced by implantation of <1 mm³ tumor sections from serially passaged PANC-1 xenograft tumors into 4-6 week old female nude mice. When the tumors reached approximately 100-200 mm³ the TfRscFv-LipA-AS HER-2 complex was i.v. injected into the tail vein daily for six days. The dose of ODN (AS or SC) administered per mouse was 10 mg/kg/injection. For comparison to standard therapy, a separate animal received chemotherapeutic agent Gemzar® (i.p.) only (60 mg/kg/injection) every other day to a total of three injections. In addition, one mouse received the combination of TfRscFv-LipA-AS HER-2 and Gemzar® at the above dose and schedule, and, as a control, one received the combination of complex carrying SC ODN and Gemzar® at the above dose and schedule. All mice were sacrificed 24 hours after the last injection and tumor, liver and lung were harvested as in Example 1. To assess tumor specific targeting in this model HER-2 and the 17 kDa cleaved caspase 3 fragment expression in the tissues was examined by Western Blot analysis Examples 3 and 4. The effect of TfRscFv-LipA-AS HER-2 on induction of the 17 kDa fragment in tumor and tissue samples also screened for HER-2 levels was examined. Western analysis as described in Examples 3 and 4, clearly shows induction of the 17 kDa fragment in the tumor from animals treated with TfRscFv-LipA AS HER-2 alone or plus Gemzar® (FIG. 11). This strong induction was not evident in the mice receiving SC ODN (TfRscFv-LipA-SC ODN) plus Gemzar® or Gemzar® alone. More importantly, this 17 kDa cleaved caspase 3 band was not evident in any of the liver or lung samples. These studies demonstrate that after intravenous administration, the complex could preferentially target and deliver the AS HER-2 ODN to tumor. In addition, the expression of the 17 kDa was evident only where the therapeutic molecule was expressed.

### Example 12

### In Vivo Chemosensitization of PanCa Xenograft Tumors by TfRscFv-LipA-AS-HER-2

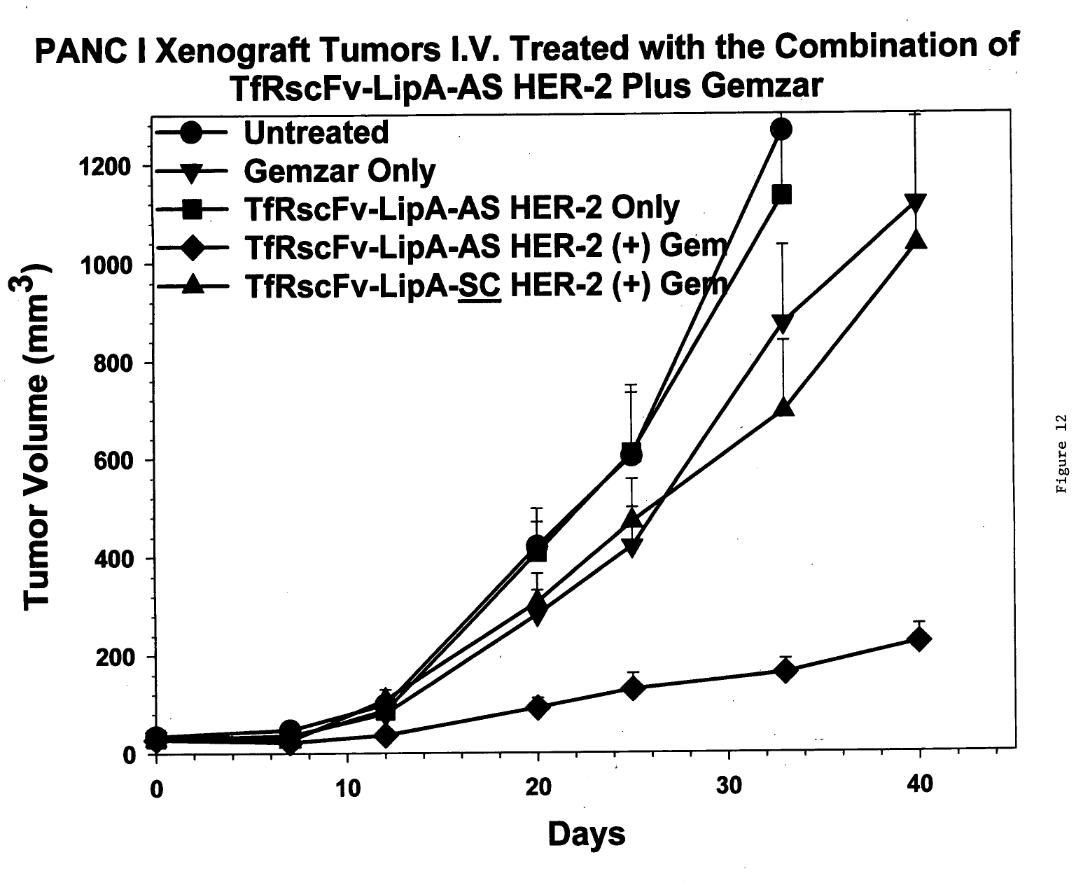

The in vitro studies described above indicated that treatment of PanCa cells with the TfRscFv-LipA-AS HER-2 complex could increase their response to Gemzar®. For this gene therapy delivery system to be clinically relevant for human cancers, e.g., PanCa, the increased sensitization observed in vitro must translate to an in vivo model. The efficacy of the TfRscFv-LipA-AS HER-2 in treating PanCa in vivo was assessed using the subcutaneous PANC-1 xenograft mouse model. Athymic nude mice (5-9 mice/group with two tumors/mouse) bearing subcutaneous xenograft tumors of ˜50 mm³ were treated three times per week with the TfRscFv-LipA-AS HER-2 complex containing ODN at 9 mg/kg/injection. As controls one group of animals received Gemzar® alone, the TfRscFv-LipA-AS HER-2 alone, or the combination of Gemzar® and the complex carrying the SC ODN. Gemzar® was given I.P. twice weekly at 60 mg/kg. The animals received a total of 18 i.v. injections of complex and 12 of Gemzar®. As shown in FIG. 12, Gemzar® alone had only minimal effect on tumor growth, while AS HER-2 only was ineffective. The groups receiving Gemzar® alone or control SC ODN plus Gemzar® are not statistically different, indicating that any growth inhibition by TfRscFv-LipA-SC ODN plus Gemzar® is strictly a drug effect. However, tumor growth was substantially inhibited in the mice that received the combination of TfRscFv-LipA-AS HER-2 and Gemzar®. The differences between the group receiving the combination therapy and Gemzar® alone or TfRscFv-LipA-AS HER-2 alone are highly statistically significant (p<0.001 by student's t-test). Thus, i.v. administration of the complex carrying AS HER-2, in combination with Gemzar®, is efficacious against PanCa.

The weights of the animals also were monitored as an indicator of toxicity. No weight loss occurred and there was no significant difference between any of the treatment groups. Thus, it appears that the TfRscFv-LipA-AS HER-2 has no major non-specific cytotoxicity. Therefore, this study clearly demonstrates that the systemically delivered, tumor-targeted liposome AS HER-2 complex can sensitize PanCa tumors to chemotherapeutic agents by inducing apoptosis as demonstrated by expression of the 17 kDa cleaved caspase 3 fragment, resulting in a more effective treatment modality.

### Example 13

### Induction of the 17 kDa Cleaved Caspase 3 Fragment by Tumor Suppressor RB94-Detection in Mouse Plasma

In vivo treatment with a different tumor suppressor gene, RB94 also has been shown to induce expression of the 17 kDa fragment of cleaved caspase 3, an indicator of apoptosis. The retinoblastoma gene RB is a tumor suppressor that encodes a nuclear phosphoprotein of 928 amino acids. The normal function of this 110-kDa protein is to repress DNA transcription and prevent cell division, thus inhibiting cell growth. (Li et. al., Cancer Research 62:4637-44, 2002; Xu et. al., PNAS 91:9837-41, 1994)

Gene replacement therapy using wild-type RB in multiple types of human cancers could suppress or reduce their tumorigenicity in vitro and in vivo. RB94 is a truncated version of RB, lacking the 112 amino acids residues at the NH₂-terminal of the full length protein with even greater efficacy than full length RB in suppressing tumor growth. The RB94 protein was found to remain hypophosphorylated longer than full length RB. Since it is the un- or hypophosphorylated form that is responsible for repression of cellular proliferation, this likely accounts for the increased potency of RB94. It has also been suggested that this N-terminal truncated RB protein also could contribute to the cellular control of apoptosis/survival (Tomei, L. D. in Apoptosis: the Molecular Basis of Cell Death, pp 279-316, 1991). Thus, delivery and expression of RB94 to tumor cells in vivo could result in induction of apoptosis. Detection of the 17 kDa fragment of cleaved caspase 3 in the plasma of tumor-bearing mice treated with RB94 would be indicative of ongoing apoptosis.

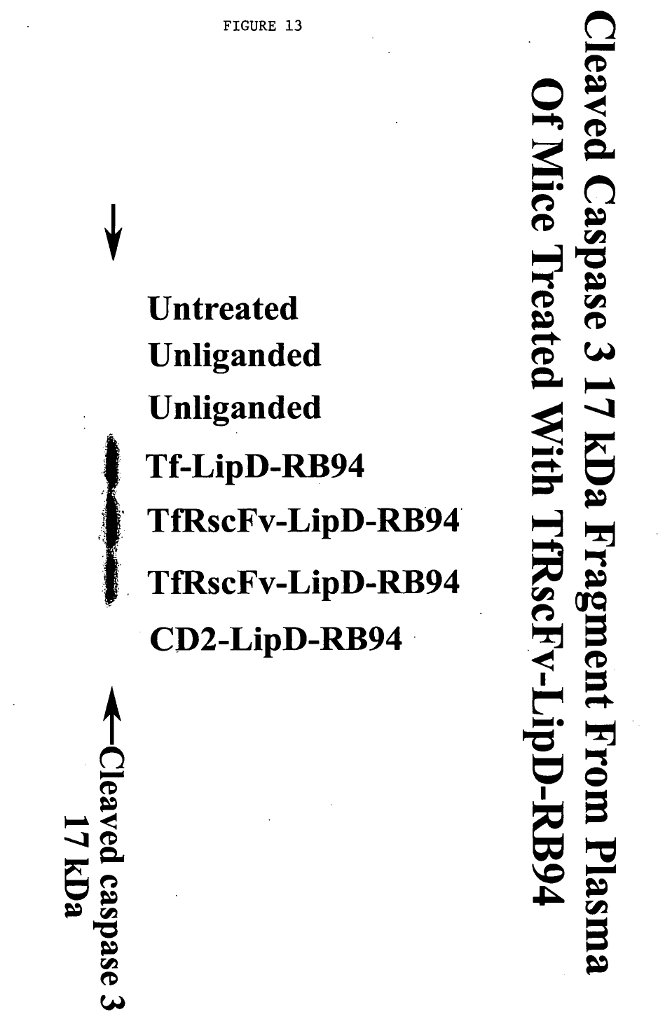

Female nude mice bearing subcutaneous xenograft tumors of human bladder carcinoma cell line HTB-9 were i.v. injected three times within 24 hours with a complex (800 μl/injection) carrying the RB94 gene (40 μg/mouse/injection). The complex also consisted of liposome D (1:1 DOTAP:cholesterol) and as a ligand, either Tf itself or the TfRscFv molecule. As controls, other mice were i.v. injected with the complex without targeting ligand, or with a non-tumor specific molecule (CD₂) as the ligand. None of these were expected to go to or affect the tumor. Sixteen hours after the last injection the animals were sacrificed, blood taken as plasma isolated as described in Example 2. Western analysis of the expression of the 17 kDa fragment of cleaved caspase 3 was performed as described in Examples 3 and 4. Forty μg of protein were run per lane of a 4-20% polyacrylamide/SDS gel. As shown in FIG. 13, the 17 kDa cleaved caspase 3 protein was only evident in the plasma from the mice receiving the RB94 complex that could target and affect the tumors. Thus, the non-invasive detection in plasma of the 17 kDa cleaved caspase 3 fragment, an indicator of apoptosis, can serve as a general pharmacodynamic marker of gene therapy. The method is broadly applicable, not simply for p53.

### Example 14

### Detection of 17 kDa Cleaved Caspase 3 Fragment in Serum from Human Breast Cancer Patients After Therapy

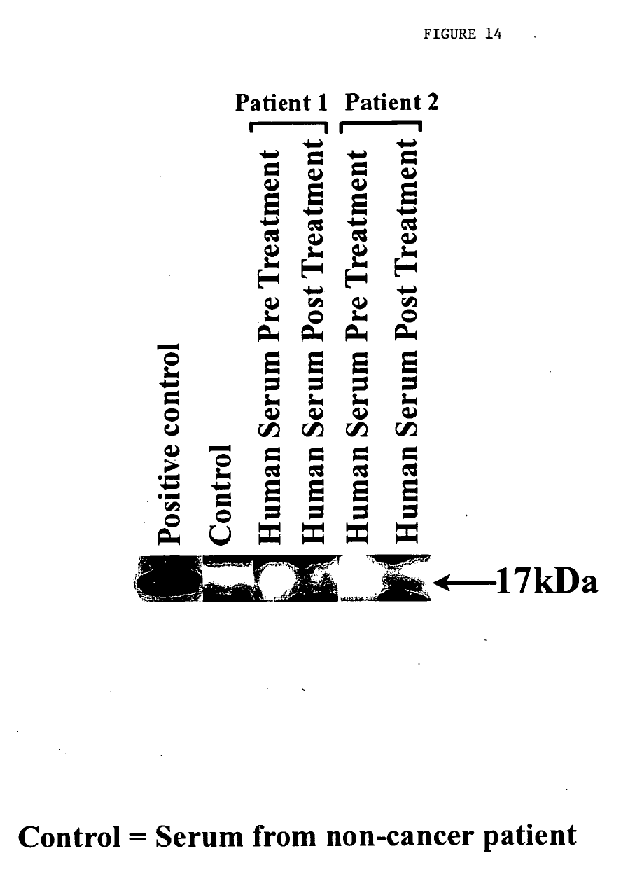

To establish that the results observed in the animal model can be applied to humans and that the expression of the 17 kDa cleaved caspase 3 fragment can be used to non-invasively assess therapeutic effect, a matched set of serum samples was obtained from two human patients who had been treated for breast cancer using conventional chemotherapy. These serum samples were obtained before (pre-) and after (post-) treatment. The serum was purified using the P6 (in Tris) Micro-Bio-Spin® Chromotography Columns. (Bio-Rad Laboratories, Hercules, Calif.) (Example 2). The flow-through from the columns was diluted at a ratio of serum to RIPA buffer of from 0.1:1, to 10:1, preferably at 1:1. Equal volumes (1 to 100 μl) were run on a 4-20% polyacrylamide/SDS gel, transferred and probed for expression of the 17 kDa cleaved caspase 3 fragment as described in Examples 3 and 4. As shown in FIG. 14, the 17 kDa cleaved caspase 3 fragment is not evident in the serum from either a control (non-cancer bearing) human subject or the patients pre-treatment. This band is clearly present, however, post-standard chemotherapy. Thus, as shown in the animal model, the expression of the 17 kDa cleaved caspase 3 fragment, an indicator of apoptosis, does correlate with cancer therapies (gene, antisense, and chemotherapy) in human patients. Thus, for any therapy that induces apoptosis, including radiation therapy, analysis of blood (as serum or plasma) for the 17 kDa cleaved caspase 3 fragment, as described in the Examples contained in this application, can be a relatively non-invasive method to monitor the effectiveness of the therapy. In human cancer patients it is envisioned that blood (1 ml to 3 ml) can be drawn in heparinized tubes and centrifuged at 300 to 1000×g, at 40 to 27° C. for 3 to 10 minutes to obtain plasma. This plasma can be run directly (as described in Examples 3 and 4) or further purified by centrifugation of a 20-75 μl aliquot of the sample through a P6 or P30 Micro Bio-Spin® Chromatography Column (preferably P6) at 300 to 2000×g (preferably 1000×g) for 1 to 10 minutes (preferably 4 minutes) at 4° to 27° C. (preferably 18-24° C., most preferably 20° C.). The flow-through is diluted with RIPA buffer at a ratio of plasma to RIPA of 0.1:1 to 10:1, preferably 1:1 before electrophoresis on a 4-20% polyacrylamide/SDS gel, transferred to any nylon or nitrocellulose solid support membrane, preferably Protran® (S+S), with a pore size of 0.1 to 0.45 μm, preferably 0.22 μm. Detection is performed using a polyclonal or monoclonal anti-caspase 3 antibody that detects the 17 kDa fragment, preferably only the 17 kDa fragment, by radioactive or non-radioactive means, preferably non-radioactive, preferably non-colorimetric, preferably via chemiluminescence, preferably enhanced chemiluminescence such as found in the ECL Western Blotting detection reagents and analysis system (Amersham Biosciences, Piscataway, N.J.), with exposure to autoradiography film including, but not limited to Hyperfilm ECL, for times ranging from 30 seconds to 24 hours, preferably 1 minute to 18 hours.

In another embodiment, serum, isolated from 1-3 ml of whole blood by collection in non-heparinized tubes and allowed to coagulate for 5-90 minutes, preferably 30-60 minutes, at 18-24° C., can be used to detect the presence of the 17 kDa fragment. The clotted sample is then centrifuged at 0.01 to 1000×g, preferably at 0.05 to 0.1×g, most preferably at 0.1×g for 0.5 to 30 minutes, preferably for 5-15 minutes, most preferably for 10 minutes and the serum removed. The serum can be analyzed directly as described in Examples 3 and 4, or purified through the same columns and analyzed by Western blot analysis in the same manner as described above for plasma.

1. A method for evaluating the efficacy of a therapeutic agent in the body of a mammal, wherein said agent acts to stimulate apoptosis, which comprises:
obtaining from a mammal to be treated with said therapeutic agent a sample of a body tissue in which tumor cells are present or a body fluid, wherein said tissue or fluid can contain a 17 kDa fragment of caspase 3, said fragment produced by specific cleavage of caspase 3 in vivo;
assaying said sample to determine the amount of said cleaved 17 kDa fragment of caspase 3 present;
administering said therapeutic agent to said mammal;
obtaining a second sample of said body tissue or said body fluid from said mammal; and
assaying said second sample to determine the amount of said 17 kDa fragment of cleaved caspase 3 present;
wherein an increase in the amount of said 17 kDa fragment measured in said second sample over the amount measured in said first sample correlates with apoptosis stimulation and efficacy of said therapeutic agent.

2. The method of claim 1, wherein said body fluid is blood or a blood component.

3. The method of claim 2, wherein said blood component comprises plasma, serum or blood cells.

4. The method of claim 1, wherein said body fluid is saliva.

5. The method of claim 1, wherein said therapeutic agent comprises a chemotherapeutic agent, a radiotherapeutic agent, a tumor suppressing nucleic acid, an oligonucleotide or a combination thereof.

6. The method of claim 1, wherein said therapeutic agent comprises a nucleic acid.

7. The method of claim 6, wherein said nucleic acid comprises a DNA molecule which encodes a wild type p53 molecule, an RB molecule, an RB94 molecule, an apoptin molecule or an antisense HER-2.

8. The method of claim 1, wherein said therapeutic agent is administered as a complex with a ligand-cationic liposome.

9. The method of claim 8, wherein said ligand comprises transferrin, folate or an anti-transferrin receptor single chain antibody fragment.

10. The method of claim 8, wherein said ligand comprises an antibody or antibody fragment.

11. The method of claim 10, wherein said antibody or antibody fragment binds to the transferrin receptor or to HER-2.

12. The method of claim 10, wherein said antibody fragment is an scFv fragment.

13. The method of claim 8, wherein said liposome comprises a mixture of dioleoyltrimethylammonium phosphate (DOTAP) and dioleoylphosphatidylethanolamine (DOPE) or cholesterol or a combination thereof or a mixture of dimethyldioctadecylammonium bromide (DDAB) and DOPE or cholesterol or a combination thereof.

14. The method of claim 8, wherein said therapeutic agent further comprises a chemotherapeutic agent or a radiotherapeutic agent.

15. The method of claim 1, wherein the amount of said cleaved 17 kDa subunit in said second sample is at least 1.5 to about 2 times the amount of said cleaved subunit in said first sample.

16. A method for evaluating the efficacy of a therapeutic agent in the body of a mammal, wherein said agent acts to stimulate apoptosis, which comprises:
obtaining a sample of blood or a blood component from a mammal to be treated with said therapeutic agent;
assaying said sample to determine the amount of a 17 kDa fragment of caspase 3 present in said sample, said fragment produced by specific cleavage of caspase 3 in vivo;
administering said therapeutic agent to said mammal;
obtaining a second sample of blood or a blood component from said mammal; and
assaying said second sample to determine the amount of said 17 kDa fragment of cleaved caspase 3 present in said sample;
wherein an increase in the amount of said 17 kDa fragment measured in said second sample over the amount measured in said first sample correlates with apoptosis stimulation and efficacy of said therapeutic agent.

17. The method of claim 16, wherein said blood component comprises plasma, serum or blood cells.

18. The method of claim 16, wherein said therapeutic agent comprises a chemotherapeutic agent, a radiotherapeutic agent, a tumor suppressing nucleic acid, an oligonucleotide or a combination thereof.
